(19) United States
(12) Patent Application Publication
(10) Pub. No.: US 2023/0039773 A1
(43) Pub. Date: Feb. 9, 2023

(54) IMPLANT FOR TREATING ANEURYSMS

(71) Applicant: femtos GmbH, Bochum (DE)

(72) Inventors: Hermann MONSTADT, Bochum (DE); Ralf HANNES, Dortmund (DE); Hans HENKES, Stuttgart (DE); Volker TRÖSKEN, Witten (DE); Ronald KONTEK, Bochum (DE); Dennis HERKLOTZ, Wuppertal (DE); Dennis GERMEROTH, Herne (DE)

(73) Assignee: femtos GmbH, Bochum (DE)

(21) Appl. No.: 17/781,844

(22) PCT Filed: Dec. 15, 2020
(86) PCT No.: PCT/EP2020/086228

§ 371 Date: Jun. 2, 2022

(30) Foreign Application Priority Data
Dec. 23, 2019 (DE) 10 2019 135 748.9
Jul. 10, 2020 (DE) 10 2020 118 301.1

(51) Int. Cl. A61B 17/12

(52) U.S. Cl. CPC A61B 17/12113; A61B 17/12172; A61B 2017/1209; A61B 2017/12063; A61B 2017/00867

(57) ABSTRACT

An implant (1) for the treatment of arteriovenous deformities, in particular aneurysms (2). In an expanded state the implant has a basic body (6) comprising of a proximal and a distal segment (7, 8), with the proximal and the distal segment (7, 8) being of dome-shaped configuration, with the convex side of the dome of the proximal segment (7) facing in the proximal direction and the convex side of the dome of the distal segment (8) facing in the distal direction, and wherein the proximal and the distal segment (7, 8) are connected to each other via a plurality of connecting struts (9). Alternatively, the implant (1) may have the shape of a closed tulip blossom. The inventive implant (1) is able to adapt well to the shape of the respective aneurysm (2).

The invention relates to an implant for the treatment of arteriovenous deformities, in particular aneurysms, with the implant being navigable in a compressed state via a microcatheter to a destination in the blood vessel system of a patient and the implant being preset to a secondary structure which causes it to assume an expanded state when released from the microcatheter, with the implant being detachably connected to an insertion aid via a severance or detachment point.

Aneurysms are usually saclike or fusiform dilatations of the vessel wall and occur primarily in structurally weakened areas of the vessel wall due to the constant pressure of blood. Accordingly, the inner vessel walls of an aneurysm are thus sensitive and susceptible to injury. As a rule, rupture of an aneurysm leads to significant health impairment, and in the case of cerebral aneurysms, to neurological deficits and even fatalities of patients.

Aside from surgical interventions, in which, for example, the aneurysm is clamped by means of a clip, endovascular methods for the treatment of aneurysms are known in particular, with two approaches being primarily pursued. One option includes filling the aneurysm with occlusion means, especially using so-called coils (platinum spirals) for this. The coils facilitate the formation of a thrombus and thus ensure occlusion of the aneurysm. On the other hand, it is known to close off the access to the aneurysm, for example the neck of an berry aneurysm, from the blood vessel side making use of stent-like implants and in this manner disconnect it from the blood flow. Both methods serve to reduce the blood flow into the aneurysm and in this way alleviate, ideally even eliminating the pressure acting on the aneurysm and thus reducing the risk of an aneurysm rupture.

When filling an aneurysm with coils it may happen that the filling of the aneurysm is inadequate, allowing blood flow into the aneurysm and in this way cause the pressure acting on its inner wall to continue. The risk of steady dilation of the aneurysm and eventually its rupture persists, albeit in an attenuated form. Moreover, this treatment method is only suitable for aneurysms having a relatively narrow neck—so-called berry aneurysms—as otherwise there is the risk that coils protrude from a wide aneurysm neck into the blood vessel where they produce clots, which may lead to occlusions in the vessel. In the worst case, a coil is completely washed out of the aneurysm and causes vessels to be occluded elsewhere. To keep the coils in place in the aneurysm sac, the aneurysm neck is often additionally covered with a special stent.

Another intravascular treatment approach focusses on so-called flow diverters. These implants are similar in appearance to stents that are used for the treatment of stenoses. However, since the purpose of the flow diverters is not to keep a vessel open, but to obstruct access to the aneurysm on the blood vessel side, their mesh size is very narrow; alternatively, implants of this kind are coated with a membrane. A disadvantage of these implants is the risk that outgoing side branches in the immediate vicinity of the aneurysm to be treated are sometimes also covered and thus closed off in the medium or long term.

WO 2012/034135 A1 discloses an implant which comprises a first and a second section, which are arranged one behind the other within a catheter, but after release within the aneurysm take on a three-dimensional, near-spherical shape and in this way fill the aneurysm. The basic material for the three-dimensional implant is a mesh-like fabric, the embodiment examples and figures are all based on a tubular braiding consisting of shape memory material. The disadvantage of this prior art example has been found to be that said implant has an unfavorable stiffness. Due to the fact that aneurysms are rarely absolutely round, a three-dimensional implant should be able to adapt to the morphology of the aneurysm in the best possible way. Furthermore, the implant is too bulky for low-caliber catheters.

Another implant for insertion into aneurysms is disclosed in WO 2017/089451 A1. The implant described therein comprises several subunits, each of which has a framework of struts with a covering arranged between them. However, the implant must first form into a plurality of coils within an aneurysm until sufficient coverage of the aneurysm surface is achieved.

WO 2009/135166 A2 discloses an implant for aneurysms that is of barrel-like shape and composed of a plurality of interwoven filaments that converge centrally at a point at the proximal and distal ends. With the help of such an implant, it is possible to close off an aneurysm using a single implant. On the other hand, the blood flow may cause the barrel-like structure to be depressed resulting in the overall volume of the implant to decrease. This is associated with incomplete filling of the aneurysm.

In general, the problem arises that on the one hand it would be desirable to have available an implant which alone is capable of filling an aneurysm. What must be borne in mind here is, however, that aneurysms are often irregularly shaped and vary in size, so a single implant should be capable of effectively adapting to these different shapes. It is precisely because of this problem that conventional coils are still used frequently, because they largely fill the space of the aneurysm independently and become entangled in the process, with the attending physician adjusting the number of coils inserted depending on the size of the aneurysm.

Proceeding from the prior art referred to hereinbefore, it is thus the objective of the invention to provide an implant which, as a single implant or in conjunction with other occlusion means, is capable of filling an aneurysm while adapting to the shape and size of the aneurysm interior.

According to a first embodiment, this objective is achieved by the proposed invention providing an implant for the treatment of arteriovenous deformities, in particular aneurysms, wherein the implant can be brought in a compressed state through a microcatheter to a target site in the blood vessel system of a patient and the implant being preset to a secondary structure causing it to assume an expanded state when released from the microcatheter, wherein the implant is separably connected to an insertion aid at a detachment point and wherein the implant in the expanded state has a basic body comprising a proximal and a distal segment, with the proximal and the distal segment being of dome-shaped configuration, with the convex side of the dome of the proximal segment facing in the proximal direction and the convex side of the dome of the distal segment facing in the distal direction, and wherein the proximal and the distal segment are connected to each other via a plurality of connecting struts.

Due to the fact that the implant has a distal and a proximal dome with connecting struts extending between them, the implant offers a high degree of flexibility. This is ensured in both axial and radial directions, that is, the implant is capable of adapting to the available space in the longitudinal direction, but also in the direction of rotation around the longitudinal axis. In the expanded state, the implant may be slightly larger than the interior of the aneurysm, but the flexibility created by the connecting struts extending longitudinally between the distal and proximal domes enables effective adaptation of the implant. For example, the connecting struts can be slightly compressed or twisted to achieve adaptation in axial or radial direction, respectively. The implant secures itself in the aneurysm due to its usually slightly excessive dimensions compared to the size of the aneurysm to be treated. In this context, the connecting struts serve to bring about length compensation and adaptation to the aneurysm. Good anchoring is achieved due to the spring effect.

Longitudinal direction shall be understood to be the direction corresponding to or parallel to the axis running from proximal to distal, with connecting struts extending in the longitudinal direction are also understood to include those that have a longitudinal component but do not run in the longitudinal direction in a straight line. It is advantageous, in fact, if the connecting struts existing between the proximal and distal segments are curvilinear, because this ensures flexibility with regard to length. The connecting struts may stretch slightly by assuming a straighter configuration, but they can also be pressed together if the curvature increases. In this way, the connecting struts exert a certain spring effect, both in the axial and radial directions. The spring effect improves anchoring of the implant in the aneurysm. A curvilinear extension is understood as a configuration of the connecting struts with one or more curvatures.

With respect to the longitudinal direction, the individual connecting struts run essentially parallel, that is, the connecting struts do not have intersections and are not interwoven with each other normally so that they can be compressed or stretched independently of each other. Similarly, rotational movement of the distal segment relative to the proximal segment about the longitudinal axis is possible, i.e. the connecting struts thus also ensure radial flexibility. As proposed by the invention, an essentially parallel configuration of the connecting struts in the longitudinal direction also exists if the extension of the curvature lines of the individual connecting struts differs.

The terms “proximal” and “distal” are to be understood in such a way that, when the implant is inserted, parts facing towards the attending physician are referred to as proximal and parts facing away from the attending physician are referred to as distal. Typically, the implant is thus moved forward in distal direction by means of a microcatheter. For example, the microcatheter may be a microcatheter having an inside diameter of 0.021″, 0.027″ or 0.033″. The term “axial” refers to the longitudinal axis of the implant, which extends from proximal to distal when the implant is in an elongated position, with “radial” referring to a direction orthogonal to this.

The dome-shaped proximal and distal ends of the basic body can also be described as plate-shaped. To enable to adjust better to the shape of the aneurysm, the segments arranged in the end region are typically slightly rounded toward their respective ends. Moreover, a rounded shape is particularly atraumatic. The convex curvature of the domes or plates thus points towards the distal or proximal end of the basic body, while the concave curvature points towards the interior of the basic body. As the case may be, the proximal and distal segments may also be beveled instead of rounded, in which case the tip, which is relatively flat typically, is understood to be the convex side. However, especially the proximal plate does not have to be completely rounded; often there is a rounding, but it has an extension centrally in the proximal direction where the proximal plate terminates toward the detachment point. The diameters of distal and proximal segments in the expanded state are variable and can appropriately adjust to specific conditions, so that different aneurysm sizes and aneurysm neck sizes can be treated. The implant is normally designed such that it would assume a diameter larger than the interior of the aneurysm during free expansion. Therefore, the implant secures itself in the aneurysm in a force-closed manner.

Expediently, the proximal and/or the distal segment are composed of frame struts which are at least partially connected to each other. These frame struts may in particular form a mesh or loop structure in the proximal and/or distal segment. The frame struts thus intersect at certain crossing points, so that gaps between the frame struts are created. These interspaces or meshes/loops may have different shapes and, for instance, can be honeycomb, diamond or flower shaped, with the edges being rounded. Rounded shapes offer advantages in that they are particularly atraumatic. However, the gaps/interspaces do not have to be completely surrounded by struts; the loops may rather be provided with an opening to one side, for example, which will further increase the flexibility of the segment. Viewed from distal or proximal, the implant may, for example, have 3 to 8 loops arranged in a circle.

With respect to its main structure, the basic body of the implant essentially is thus preferably composed of frame struts forming the proximal and distal segments, and connecting struts, which connect the proximal and distal segments to one another. The design of the struts makes sure the implant is easily folded and unfolded for placement in or release from the microcatheter.

The connecting struts link the proximal and distal segments. The total number of connecting struts can be, for example, 3 to 10, in particular 6 to 8. The connection points between the connecting struts and the distal or proximal segment, which often consists of frame struts, can be arranged in various ways and, for example, may be arranged in one plane, but can also be offset from one another. The length and shape of the connecting struts may also be the same, but can also be different. In the case of staggered connecting points, for instance, it may also be necessary to arrange connecting struts of different length. For the connecting struts different cross- sections can also be selected as required for the intended use.

In particular, the struts (frame struts and connecting struts) of the implant can be produced by laser cutting techniques. However, it is also possible to provide a braided structure for the basic body, with individual struts being intertwined or woven together. Other manufacturing processes may be adopted as well, such as galvanic or lithographic production, 3D printing or rapid prototyping. The struts provided may have a round, oval, square or rectangular cross section, with the edges may have a rounded off configuration in the event of a square or rectangular cross section. The individual struts can also be made up of several individual filaments that are twisted together or extending in parallel.

Expediently, the distal segment may include an area free of frame struts, said area being expandable and compressible. This area is preferably located centrally in the distal segment, so that it is arranged approximately at the distal end of the basic body. In the event of the preferred mesh/loop structure, an opening is thus created in the distal segment between the meshes/loops. This area results in the size of the distal segment to be adapted to the requirements within the aneurysm, especially with regard to the circumference. To satisfy the specific application needs, different sizes and a different number of meshes/loops can be selected. As the size and shape of the aneurysm determine, the distal segment may dilate or be compressed to a greater or lesser extent, with flexibility being achieved via the (central) region devoid of frame struts. This area thus creates a certain amount of resilience or spring effect.

It is especially preferred to provide the proximal segment with a membrane that covers the proximal segment at least partially, with said membrane thus covering the aneurysm neck and largely prevents the inflow of blood into the aneurysm. By disconnecting the aneurysm from the flow of blood, it eventually atrophies/degenerates and the risk of rupture of the aneurysm is eliminated, with the membrane thus having a blood flow modulating effect.

The membrane need not be necessarily confined to the proximal segment; a membrane may as well be arranged on the distal segment, and/or the connecting struts could also be provided with a membrane. In case there is an area in the distal segment where no frame struts are arranged as described hereinbefore, this area should also be free of the membrane. Arranging additional membranes will enhance the blood flow modulating effect of the membrane(s). Moreover, risks associated with an endoleak, i.e. the inflow of blood through a leak between the implant and the aneurysm sac, are further reduced.

In accordance with a second embodiment, the invention relates to an implant for the treatment of arteriovenous deformities, in particular aneurysms, with the implant being navigable in a compressed state via a microcatheter to a destination in the blood vessel system of a patient and the implant being preset to a secondary structure which causes it to assume an expanded state when released from the microcatheter, with the implant being detachably connected to an insertion aid via a severance or detachment point and, in the expanded state, having a basic body which is composed of struts, the struts being at least partially connected to one another at points of intersection, so that interspaces are created between the struts, with the struts, in the expanded state, extending radially outwards at the proximal end of the basic body and, in the further course, extending axially in the distal direction and radially inwards, so that bulging of the basic body is produced, with the basic body having a zone, at the distal end, where the struts do not have any connection to one another. In the expanded state, the basic body may in particular have an opening at the distal end.

According to this second embodiment, the basic body in the expanded state has the shape of a closed tulip blossom. The struts converge centrally at the proximal end at the detachment point. Starting from the detachment point, the struts first run outward in radial direction and then further on distally, at the same time approaching the central longitudinal axis again. In some circumstances, the struts may also run in the proximal direction in the section where they extend outward in the radial direction, resulting in an inward curvature at the proximal end for the basic body as a whole. The struts are at least partially connected to one another, so that intermediate spaces or meshes are created between the edges of the struts, said spaces or meshes can be of different shape, for example have a honeycomb, heart, leaf or diamond shape, with the edges of the intermediate spaces/meshes usually being rounded. Interconnected struts may form segments resembling the petals of a tulip, that is, several petal-like segments emanate from the proximal end of the basic body and form the outer surface of the basic body distributed over the circumference, said segments abut the inner wall of the aneurysm. The segments converge at the proximal end of the basic body. In this way, a basic body is created in which the individual segments are elastic and movable so that they are capable of adapting well to the shape of the aneurysm, resulting in atraumatic implantation. The elastic distal ends of the struts/segments serve as a support to push the proximal end of the basic body into the aneurysm neck, promoting a good seal of the aneurysm to the blood stream.

At the distal end, the struts are not connected to each other so that the basic body is open here. The struts or the segments formed by the struts, however, may overlap at the distal end, so that the opening is virtually closed off, but the opening at the distal end may remain if each of the struts/segments end distally in front of the opening.

In general, the basic body largely adjusts to the shape of the aneurysm, that is, the struts first extend radially outward to the aneurysm wall and then continue along it further distally forming a radially inward curvature. The exact shape that the basic body forms inside the aneurysm thus depends as well on the shape of the aneurysm. Whether an opening remains at the distal end of the basic body or the struts overlap also depends on the shape of the aneurysm. In case of a rather small aneurysm relative to the main body, the struts may, for example, be compressed to the point where their distal ends overlap and the opening disappears, whereas in a larger aneurysm a distal opening may remain. In the expanded state, the implant may be slightly larger than the interior of the aneurysm, but the flexibility created by the struts enables an effective adaptation of the implant. The implant secures itself in the aneurysm due to its usually slightly excessive dimensions compared to the size of the aneurysm to be treated. The shape of the basic body resembles that of a closed tulip blossom, but can also be described as an upset spherical or ellipsoidal shape, although this is understood to mean not exclusively the exact geometric shape, but also a shape that at least approximates the relevant geometric shape. Generally, an expanded basic body flattened at the proximal end or having an inward invagination centrally is useful to prevent portions of the implant from reaching out of the aneurysm into the parent blood vessel.

With regard to the struts for the second embodiment of the invention, what has been stated about the first embodiment applies, that is to say, the struts of the implant may be produced in particular by laser cutting techniques, but it is also possible to have a basic body in the form of a braided structure comprising individual struts which are braided or woven together. Other manufacturing processes may be adopted as well, such as galvanic or lithographic production, 3D printing or rapid prototyping. The struts provided may have a round, oval, square or rectangular cross section, with the edges may have a rounded off configuration in the event of a square or rectangular cross section. The individual struts can also be made up of several individual filaments that are twisted together or extending in parallel.

Similarly, in accordance with the second embodiment, one or more membranes covering the basic body may be provided. It is particularly preferred to provide the proximal area of the basic body with a membrane that enables an at least partial covering. As a result, the membrane covers the aneurysm neck and largely prevents the inflow of blood into the aneurysm. Disconnecting the aneurysm from the flow of blood causes it to eventually atrophy/degenerate and the risk of rupture of the aneurysm is eliminated, with the membrane thus having a blood flow modulating effect.

The membrane does not have to be limited to the proximal area of the basic body; also the distal area can be provided with a membrane. Nevertheless, free of membranes is normally the distal end where there is an opening if the distal ends of the struts do not overlap too much. The blood flow modulating effect of the membrane(s) is intensified by providing a membrane in additional areas of the basic body. Moreover, risks associated with an endoleak, i.e. the inflow of blood through a leak between the implant and the aneurysm sac, are further reduced.

Irrespective of the embodiment, the implant is preferably made at least in part of shape memory materials. This enables the desired secondary structure to be preset to the implant, which it automatically assumes when leaving the microcatheter. In the field of medical engineering, shape memory metals are particularly well known, with nickel-titanium alloys, such as those used under the name Nitinol, deserving special mention in this respect. As a rule, the secondary structure is created upon exiting the microcatheter as a result of the elimination of the external constraint exerted by the microcatheter; however, it is also possible that the secondary structure forms as a result of the temperature change that occurs when exiting the microcatheter.

Via a detachment/severance point the implant is detachably connected to an insertion aid. This insertion aid may be a conventional pusher wire used to advance the implant through the blood vessel system to the desired site. However, an insertion aid that is of tubular or hose-shaped design and has an inner lumen is particularly preferred. With the help of such an insertion aid, after the basic body has been inserted into the aneurysm and expanded there, additional occlusion means can be inserted into the aneurysm and/or the interior of the basic body, by means of which an additional occlusion of the aneurysm is achieved. It is also conceivable to use the insertion aid for introduction of other filler materials into the aneurysm that are known from prior art such as viscous embolizates such as onyx.

An additional filling with other occlusion means or materials is advantageous in that a compression (compacting) of the implant by external forces is prevented. In practice, it may happen that the aneurysm acts from the outside on the inserted implant, which is hollow on the inside, so that the implant is compressed and is no longer able to completely fill the aneurysm, or is partially squeezed out of the aneurysm, which would cause obstruction of the flow of blood in the parent vessel. This is prevented by additionally filling the interior of the implant with occlusion means or materials introduced through the tubular or hose-shaped insertion aid.

Furthermore, additionally inserted occlusion means can fill gaps between the exterior of the implant and the inner wall of the aneurysm. This is particularly important when treating irregularly shaped aneurysms. To allow the additionally introduced occlusion means, especially conventional occlusion coils, to enter the space between the implant exterior and the aneurysm inner wall, the distal segment or the distal area of the basic body should not be covered by a membrane; instead, the membrane, if provided, should be confined to the proximal segment or the proximal area. It is considered particularly advantageous if, when the basic body is expanded, the tubular or hose-shaped insertion aid ends in the latter approximately in the middle, which enables the occlusion means introduced through the insertion aid to migrate outwards from there and pass through the interstices between the struts into the region between the implant and the aneurysm.

If the insertion aid has an inner lumen, the insertion of additional occlusion means or occlusion material is significantly simplified. In the case of implants that are provided with membranes, there is in fact the question of explorability, that is, to detect how additional occlusion means or materials can be introduced into the aneurysm to achieve as complete a seal as possible against the inflow of blood from the parent vessel. If a subsequent insertion is undertaken, it may be necessary to introduce another catheter and pierce the membrane of the implant. As proposed by the present invention, this is significantly simplified because the insertion of occlusion means or materials can already be accomplished during implantation of the implant. Preferably, the implant is first expanded in the aneurysm, but not yet detached. Occlusion means are then introduced through the tubular or hose-shaped insertion aid. Finally, detachment and thus final liberation of the implant takes place so that the insertion aid and microcatheter can be withdrawn in a proximal direction.

An insertion aid provided with an internal lumen can be achieved in a variety of ways, but adequate flexibility should of course be provided to allow the insertion aid to navigate through narrow lumened blood vessels, particularly those found intracranially. For example, said insertion aid may be of catheter type design, i.e. it may consist of a braiding of metal and/or plastic material. However, the insertion aid may as well be designed as a tube-like hypotube. For example, the tubing can be made of a flexible polymer material or a metal helix, and in the case of a metal helix, multiple layers could be provided, typically a maximum of 4 layers, preferably 2 layers. The layers thus form a hollow structure, built up from one or more spirals. In the event of several layers of metal helices, the helix configuration of two adjacent layers is preferably opposite, i.e. a right-hand helix is situated over a left-hand helix or vice versa. The helices are thus twisted in opposite directions. It is viewed useful to provide several oppositely configured layers to enable the tube to transmit torsional movements from proximal to distal.

In the case of manufacturing the tube from a polymer material, slits can be provided in the tube to further increase the flexibility of the hose.

For both a wire-shaped and a tube-shaped or hose-like insertion aid, it is advantageous if the flexibility increases from proximal to distal. This ensures that, on the one hand, the insertion aid is sufficiently rigid in the proximal region to allow advancement and also transmission of torsional forces, but on the other hand, the insertion aid is flexible enough in the distal region to be capable of navigating also through narrow blood vessels. For example, the diameter may decrease from proximal to distal, and the decrease may be gradual or occur in one or more steps.

It is also possible to construct the insertion aid from several components. Especially, in the event of a tubular or hose-like insertion aid, the proximal area can be designed as a tubular hypotube and the distal area as a flexible catheter.

Regardless of the way the insertion aid is made, it is brought to the target position by means of a microcatheter. This also applies if the insertion aid itself is of catheter-like design. Moreover, an external friction-reducing coating of the insertion aid is considered expedient, for example using PTFE (polytetrafluoroethylene, Teflon).

Detachment of the implant from the insertion aid takes place electrolytically, thermally, mechanically or chemically. The electrolytic detachment method provides for the detachment point to be electrolytically corroded by applying a voltage causing the implant to disconnect from the insertion aid. To avoid anodic oxidation of the implant, it should be electrically isolated from the detachment point and the insertion aid. The electrolytic detachment of implants is well known practice in the state of the art, especially for coils used for the purpose of closing off aneurysms. Relevant severance/detachment points are described, for example, in publication WO 2011/147567 A1. The principle is based on the fact that when a voltage is applied, a suitably designed detachment point made of a suitable material, in particular metal, is dissolved usually by anodic oxidation at least to such an extent that the areas of the implant located distally to the corresponding detachment point are released. The severance point can be made, for example, of stainless steel, magnesium, magnesium alloys or a cobalt-chromium alloy. A particularly preferred magnesium alloy is Resoloy®, which was developed by the company MeKo from Sarstedt/Germany (cf. WO 2013/024125 A1). It is an alloy consisting of magnesium and, inter alia, of lanthanides, in particular dysprosium. Another advantage of using magnesium and magnesium alloys is that magnesium residues remaining in the body are physiologically unproblematic.

The dissolution of the detachment point is brought about by applying an electrical voltage. This can be either alternating current or direct current, with a low current intensity (<3 mA) being sufficient. The detachment point in this case usually functions as anode, the metal of it being oxidized and dissolved. It is important for the detachment point to be electrically connected to a voltage source, in particular via the insertion aid. For this purpose, the insertion aid itself must also be of electrically conductive design. Due to the fact that the corrosion-inducing current is influenced by the surface of the cathode, said cathode surface should be significantly greater than the surface of the anode. To a certain extent the speed at which the detachment point is dissolved can be controlled by appropriately sizing the cathode surface in relation to the anode surface. Accordingly, the invention also relates to a device comprising a power source and, where applicable or appropriate, an electrode to be placed onto the body surface.

In particular, the detachment point may have the form of a detachment element arranged on the outside of the insertion aid and connected to the proximal end of the basic body. Applying the electrical voltage causes the connection between the detachment element and the basic body to corrode to such an extent that detachment/severance and liberation of the implant is achieved. Preferably, the detachment element is arranged annularly around the insertion aid so that said element can form a disc with a central hole through which the insertion aid can be passed. One or more detachment wires can be attached to the detachment element, said wires expediently carry insulation in order to allow the electrical voltage to be applied to the detachment point in as concentrated a manner as possible. The detachment wire(s) may be routed outside the insertion aid or through the interior of the insertion aid. If the insertion aid is made of metal, especially in case it has a catheter-like design comprising a metal braiding, the current can also be supplied via said metal braiding.

As already mentioned, it is advantageous if the insertion aid projects into the basic body in the expanded state. In particular, the tubular or hose-shaped insertion aid may extend distally beyond the detachment point. In the embodiment described hereinbefore which provides for a detachment element annularly surrounding the insertion aid, said detachment element is thus located further proximal than the distal end of the insertion aid. Accordingly, the basic body can first be introduced into the aneurysm and be caused to expand by withdrawing the microcatheter. Additional occlusion means, in particular coils, can then be introduced into the interior of the basic body via the insertion aid projecting into the basic body. Once this process is complete, implant detachment occurs and the microcatheter and insertion aid are finally withdrawn and removed from the blood vessel system.

In the event of a mechanical detachment/severance, there is typically a form-, force-closed or friction-fit connection, that is broken when the implant is liberated causing the implant to be separated from the insertion aid. One option is to provide in the distal area radial protrusions on the outside of the insertion aid, with said distal area projecting into the implant. The microcatheter is positioned so as to surround the insertion aid. In this way, a friction-fit connection is established between the insertion aid, the proximal end of the implant and the microcatheter. When the microcatheter is retracted in a proximal direction relative to the implant and the insertion aid, the external constriction induced by the microcatheter is eliminated resulting in the proximal end of the implant to expand radially and be freed from the protrusions, that is, the friction fit is disengaged.

In this context, the distal area of the insertion aid is understood to be a region situated further distally that interacts with the implant, but it does not have to be the distal end of the insertion aid, which often extends even further into the implant to enable placement of occlusion means at this point. Radial protrusions are those that project outward in a radial direction, i.e., orthogonal to the longitudinal axis of the insertion aid.

The external constraint preventing the proximal end from expanding must not always be brought about by the microcatheter; it is also possible to provide an additional sheath surrounding the distal end of the insertion aid, which presses the proximal end of the implant onto the insertion aid. To enable liberation to take place, this sheath must of course also be retractable in the proximal direction relative to the insertion aid and implant.

The radial protrusions can consist of an elastic material, in particular may be of elastomeric material. The proximal end of the implant is pressed by the microcatheter or the sheath onto the insertion aid and virtually clamped in place, so that a friction-fit connection is achieved. In particular, the protrusions can be shaped as pads. Said pads can surround the insertion aid in annular form, with several radially extending rings or a helix can be provided to bring about a particularly secure fastening.

The proximal end of the implant may as well be provided with thickenings, which, for example, are held between the radial protrusions of the insertion aid. For example, the thickenings may be of spherical shape or have a similar geometric form. In this case, the fixation of the implant to the insertion aid is a combination of friction- and form-closed fit.

To visualize the detachment for the attending physician, radiopaque markers located in the area where the implant is attached to the insertion aid are of advantage. For example, in this area the insertion aid, the proximal end of the implant and/or the microcatheter resp. a sheath surrounding the insertion aid can be provided with radiopaque markers whose movement, in particular their movement relative to each other, can be observed by radiographic techniques.

Also possible is the provision of a purely form-closed fastening method. In this respect, detachment elements may be arranged at the proximal end of the implant, said elements engaging in a form-closed manner with recesses provided for this purpose in the tubular or hose-shaped insertion aid. Due to the fact that the insertion aid is surrounded by the microcatheter or an additional sheath, the detachment elements are prevented from expanding radially and exiting the recesses. This makes sure the detachment elements are held securely in the recesses as long as there is no retraction of the microcatheter or sheath in the proximal direction. Only when the microcatheter/sheath is moved proximally and no longer covers the recesses can the detachment elements exit the recesses, resulting in detaching and finally liberating the implant. The recesses in the insertion aid may encompass the entire wall of the insertion aid or merely be recessed into the wall as indentations.

According to a further variant, one or more detachment elements extend in the proximal direction at the proximal end of the implant and are held in place in a form-closed manner by one or more retaining elements arranged on the insertion aid. The retaining element is made of a material having shape memory properties, with the retaining element being preset to assume a secondary structure which it strives to assume but is prevented from occupying as long as a microcatheter or separate sheath surrounds the retaining element. The external constraint may in particular prevent the retaining element from occupying the secondary structure; however, it is also possible for the retaining element to undergo a change in temperature after the microcatheter/sheath has been retracted, causing said element to assume its secondary structure. When assuming its secondary structure, the retaining element opens and releases the detachment element, that is, resulting in the implant being detached.

For example, in this embodiment, the detachment element may be of spherical shape and the retaining element may embrace the detachment element in a shell-like manner. When the shell-shaped retaining element expands, the spherical detachment element can exit. The detachment may be effected for a single detachment element interacting with a single retaining element, but it is also possible to bring together, preferably along the circumference of the insertion aid, a plurality of retaining elements with relevant detachment elements to ensure uniform release over the entire circumference.

Regardless of whether an electrolytic or mechanical detachment is involved, the proximal end of the implant retained at the detachment point may in particular be provided in the form of proximal wires, the connection of which to the detachment element/detachment point being released for the purpose of disconnecting the implant. Particularly in the case of a mechanical detachment, further detachment elements may be attached to the proximal wires, which are secured by the insertion aid.

Another option is to design the detachment points as thermal detachment points. In the event of a thermal detachment point, the connection between longitudinally adjacent sections of the implant can be broken by heating the detachment point, causing it to soften or melt so that a detachment is effected. Another option is to make use of chemical severance in such a way that the detachment is brought about by a chemical reaction occurring at the point of detachment.

The different types of detachment, for example electrolytic and mechanical detachment, can also be combined. For this purpose, a mechanical connection, in particular brought about by a form fit, is established between the elements, and this connection remains in place until an element maintaining the mechanical connection is electrolytically corroded.

As regards a combination of electrolytic and mechanical detachment, one option is to provide an insertion aid end piece at the distal end of the insertion aid, said end piece having an electrolytically corrodible detachment point, with the insertion aid end piece being connected to an implant end piece at the proximal end of the implant via an insulating element. In particular, there may be a form-closed connection between the insertion aid end piece, the insulating element, and the implant end piece to ensure a secure attachment is achieved between the insertion aid and the implant during advancement via the microcatheter. In addition, the form-closed connection can also ensure torsional movements are appropriately transmitted.

The insertion aid end piece is preferably at least partially made of a metal that ensures electrical conductivity to the detachment point, that is, a current can be applied to the insertion aid that is conducted up to the detachment point. In order to be able to apply a current at the detachment point, it is also conceivable to provide separate conductors that are insulated from the insertion aid itself. The detachment point is normally used as anode in order to cause oxidation of the metal employed for the detachment point and thus bring about the dissolution of the detachment point. The cathode is usually an electrode arranged on the patient's body surface; however, it is also possible to provide another electrical conductor on or through the insertion aid to close the electrical circuit acting on the detachment point. Preferably, at least the detachment point is made of a metal that dissolves well electrolytically or galvanically, such as a cobalt-chromium alloy; other stainless metal alloys are also possible, however. Optionally, the detachment point can be subjected to pre-corrosion to improve its dissolution characteristics.

Also the implant end piece is made of metal in most cases. It is of advantage to manufacture the implant from a metal having shape memory properties, such as nickel-titanium alloys in particular, or from a metal such as magnesium, which dissolves relatively quickly in the body, so that after some time areas of the implant only required for insertion but not permanently will disappear. The more distally situated areas of the insertion aid end piece, which also remain in the body after detachment, may as well be made of magnesium, for example. Therefore, foreign bodies that are not required will dissolve.

The insulating element arranged between the insertion aid and implant end pieces ensures that the implant is insulated from the applied electrical voltage, thus causing the current to be focused on the detachment point. The insulating element is made of an electrically insulating material. A form fit between the two end pieces is preferably achieved/transmitted via the insulating element.

In accordance with a particularly preferred embodiment, both the insertion aid end piece and the implant end piece comprise a first short tubular element at which a narrow connecting web is arranged and extends in the longitudinal direction towards a second short tubular element which is provided with an interruption/cutout on its circumference. Preferably, this interruption/cutout is located opposite the connecting web when viewed radially. The insulating element is adapted to the two end portions in such a way that the respective second tubular elements with their cutouts can each engage with a portion of the insulating element, with the two portions of the insulating element being arranged one behind the other in the longitudinal direction. The two sections each provide space to accommodate the second tubular elements. In this context, the longitudinal direction is understood as the direction from proximal to distal or vice versa.

When the insertion aid end piece, insulating end piece, and implant end piece are joined together, the second tubular element of the implant end piece is situated proximal to the first tubular element, while the second tubular element of the insertion aid end piece is disposed distal to the first tubular element. The second tubular element of the implant end piece engages in the accommodation space of the further proximally located portion of the insulating element, whereas the second tubular element of the insertion aid end piece engages in the accommodation space of the further distally arranged portion of the insulating element. Accordingly, the insertion aid end piece and the implant end piece ultimately engage with each other, but always via an interface layer formed by the insulating element. In the assembled configuration, the second tubular element of the implant end piece is located proximal to the second tubular element of the insertion aid end piece. The interruptions/cutouts in the second tubular elements of the end pieces are generally located in radially opposite positions.

The detachment point is expediently located in the area of the narrow connecting web of the insertion aid end piece, for example where the connecting web of the insertion aid end piece is connected to the first, continuous tubular element. The detachment point is easily corrodible and dissolves by application of a voltage at least to such an extent that areas of the insertion aid end piece, that is, the first tubular element, separate from the other portions of the detachment element. In this context, the term detachment element is understood to denote the entirety of the insertion aid end piece, insulating element and implant end piece. The implant together with the implant end piece, insulating element and the further distal areas of the insertion aid end piece thus remain in the body, while the proximal area of the insertion aid end piece remains connected to the insertion aid and is removed from the body.

The advantages of the detachment element described, which represents a combination of mechanical connection via form-closed locking with electrolytic detachability, lie in particular in the mechanical safety of the connection, which, for example, is also provided when the insertion aid is withdrawn in proximal direction. A mechanical connection continues to exist even after pushing out of the microcatheter or when the microcatheter is withdrawn. Separation occurs only and in a well controllable manner when an electrical voltage is applied to the detachment element.

Basically, the detachment elements described above can also be used for the insertion of any other implants. This is particularly true for the last described embodiment, which refers to a combination of electrolytic detachment and mechanical connection and is particularly safe to use therefore. Thus, the invention encompasses corresponding detachment systems, even if the implant itself has a design that does not conform to the designs and configurations detailed in this patent application.

The detachment point may be located proximal to the basic body, but it is preferred to provide a detachment point within the basic body or implant as described, which results in the insertion aid to extend into the implant in the expanded state. On the one hand, this makes it easier to insert further occlusion means in the event the insertion aid is of a tubular or hose-shaped design, and on the other hand, the struts forming the proximal segment or the proximal area can at least partially converge at the detachment point, causing a slight invagination/inward curvature to occur in the proximal segment/area. An invagination in the proximal or also in the distal segment/area is associated with the advantage that potentially traumatic strut ends are ruled out or cannot come into contact with the aneurysm wall because they are relocated to the interior of the basic body.

A membrane covering shall be understood to mean any provision of the corresponding struts or segments with a membrane, regardless of whether the membrane is applied to the outside or inside of the struts/segments or whether the struts/segments are embedded in the membrane, with the latter being preferred.

In case the implant has been provided with a membrane, it is capable of separating and occluding the aneurysm from the bloodstream on its own, that is, without the need of additional occlusion means such as coils or similar. The membranes offer sufficient elasticity so that the folding and unfolding of the implant is not impeded and the membranes are not damaged in the process. A membrane arranged in the proximal segment or area of the implant is particularly advantageous, because in this way it creates a seal against the parent blood vessel. It is often sufficient to only insert a single appropriate implant into the aneurysm, but several implants as proposed by the invention may also be employed. Furthermore, as described above, occlusion means or occlusion material can be placed into the implant. In this context, an insertion aid having an inner lumen is of special advantage. A stent or flow diverter may also be placed in the parent blood vessel.

However, also conceivable are embodiments of the invention which provide for the basic body not to be fitted with a membrane. In this case, the implant usually serves as a supporting structure for retaining other implants such as coils, inventive implants of smaller size with or without membrane, or even other occlusion means as described in the introductory section. It is also conceivable to make use of the implant proposed by the invention in conjunction with other filler materials as they are known from the prior art, e.g. viscous embolizates such as onyx. Unless further occlusion means or materials are already introduced at the time of implantation of the implant, insertion of the implant into the aneurysm normally takes place first, followed by a microcatheter being inserted between the struts forming the implant and occlusion or embolization agents being introduced into the aneurysm through the microcatheter.

Insofar as in the framework of the present invention reference is made to membranes in the plural, it is hereby expressly pointed out that there need not be any separation between the individual membranes; rather, the individual membranes may well merge into one another and thus form an overall membrane. Conversely, a membrane, for example the membrane covering the proximal segment or the proximal area, may also be composed of several individual membranes, each of which, for example, being designed to cover/fill a mesh/loop in the proximal segment.

Within the meaning of the present invention, a membrane is a thin structure having a planar surface, regardless of whether said structure is permeable, impermeable or partially permeable to liquids. However, to accomplish the objective of the aneurysm treatment, membranes are preferred that are completely or at least substantially impermeable to fluids such as blood. Moreover, a membrane may also be designed to comprise pores through which further occlusion means can be introduced. Another option is to have the membrane designed in such a way that it can be pierced with a microcatheter for the introduction of further occlusion means or even with the occlusion means themselves.

The membranes can be made of polymer fibers or films. Preferably, the membranes are produced by an electrospinning process. In this process, the struts are normally embedded in the membrane. This can be achieved by spinning or braiding fibers around the struts, in particular the frame struts of the proximal and/or distal segment.

In electrospinning, fibrils or fibers are separated from a polymer solution and deposited on a substrate by applying an electric current. Said deposition causes the fibrils to agglutinate into a non-woven fabric. Usually, the fibrils have a diameter ranging between 100 and 3000 nm. Membranes created by electrospinning have a very uniform texture. The membrane is tenacious, withstands mechanical stresses, and can be pierced mechanically without an opening so created giving rise to cracks propagating from it. Another advantage of electrospun membranes is that they enable to establish a large contact surface with the blood. The thickness of the fibrils as well as the degree of porosity can be controlled by selecting process parameters as appropriate. In the context of producing the membrane and with respect to materials suitable for this purpose, special attention is drawn to publications WO 2008/049386 A1, DE 28 06 030 A1and literature referred to therein.

In lieu of electrospinning, the membranes may also be produced by an immersion or spraying process such as spray coating. As regards the material of the membranes, it is important that they are not damaged by the mechanical stresses arising when the implant is drawn into a microcatheter, deployed, unfolded, etc. To ensure this, the membranes should have sufficient elasticity.

The membranes can consist of a polymer material such as polytetrafluoroethylene, polyester, polyamides, polyurethanes or polyolefins. Especially preferred are polycarbonate urethanes (PCU), in particular electrospun polycarbonate urethanes. In particular, an integral connection of the membranes with the struts/segments is desirable. Such an integral connection can be brought about by covalent bonds provided between the membranes and the struts/segments. The formation of covalent bonds is promoted by silanization of the struts/segments, that is, by a chemical bonding of silicon, in particular silane, compounds to at least portions of the surface of the struts/segments. On surfaces, silicon and silane compounds attach, for example, to hydroxy and carboxy groups. Basically, aside from silanization, other methods of mediating adhesion between the struts/segments and membranes are also conceivable.

Silane compounds in this context are to be seen as all those compounds which follow the general formula RₘSiXₙ (m, n=0-4, where R stands for organic radicals, in particular alkyl, alkenyl or aryl groups, and X stands for hydrolyzable groups, in particular OR, OH or halogen, with R=alkyl, alkenyl or aryl). In particular, the silane may have the general formula RSiX₃. Moreover, relevant compounds having several silicon atoms also count among the silane compounds. In particular, silane derivatives in the form of organosilicon compounds are regarded as silane compounds in this context.

Additional substances promoting thrombogenization or endothelial formation may be embedded in or deposited on the membranes. Substances that promote thrombogenization are therefore advantageous because they support the formation of a thrombus or clot within the aneurysm, which ensures permanent occlusion of the aneurysm. An example in this context are nylon filaments. Because aneurysms are due to degenerative diseases of the vascular wall, particularly atherosclerosis, promoting endothelial formation and correcting endothelial dysfunction may also have beneficial effects. This applies especially to the area where the aneurysm is in contact with the flow of blood in the actual blood vessel (parent vessel). Preferably, substances promoting thrombogenization are applied to the inner side of the membrane, whereas substances promoting endothelial formation are applied to the outer side of the membrane, with outer side being understood here to denote the side of a membrane facing the vessel wall in the implanted state and the inner side being understood to mean the side of a membrane facing the interior of the aneurysm. Examples of substances that promote thrombogenization include collagens, whereas, for example, hyaluronic acid, statins (3-hydroxy-3-methylglutaryl coenzyme A reductase inhibitors), and other polymers may promote endothelial cell colonization. Polysaccharides, especially glycosaminoglycans, which are able to mimic the glycocalyx, are particularly suitable polymers. Another material that can be used is POSS-PCU (polyhedral oligomeric silsesquioxane poly(carbonate-urea) urethane). It is a nanocomposite that has been described, among other applications, as a scaffold for artificial organs and as a coating for medical devices, (Tan et al., Crit Rev. Biomed Eng. 2013; 41(6): 495-513). It is also possible to use POSS-PCL (polyhedral oligomeric silsesquioxane poly(caprolactone-urea) urethane). Applicable to both POSS-PCU and POSS-PCL is that in particular functionalized derivatives of these nanocomposites can also be employed. This applies in particular to those derivatives that can be obtained by linking with polyacrylic acid (poly-AA). As POSS-PCU and/or POSS-PCL nanocomposite polymers are only poorly suited for direct immobilization on the surface of an implant, it has been found advantageous to combine polymers such as polyacrylic acid (poly-AA) with the nanocomposite. This can be achieved, for example, by plasma polymerization of acrylic acid. A poly-AA-g-POSS-PCU surface obtained in this way promotes collagen bonding (especially collagen type 1) and thus endothelial formation (cf. Solouk et al., Mater Sci Eng C Mater Biol Appl. 2015; 46: 400-408). Some additives, for example collagen or hyaluronic acid, are also advantageous because they can improve the friction against the inside of a catheter during advancement and the biocompatibility of the implant. In general, biofunctional or bioactive coatings may be present on the membrane.

Preferably, the basic body in the expanded state has, by approximation, a spherical shape, an ellipsoidal shape, ovoid shape or the shape of a cylinder with convex outwardly curved base surfaces. In particular, in the case of the second embodiment, the shape of the basic body in the expanded state can also be described as a closed tulip blossom. In this context, the specified geometric shape is understood to be not only the exact shape, but also a shape that at least approximates the respective geometric shape. Basically, a spherical shape is well suited to fill a regularly shaped aneurysm. However, when the implant is compressed or stretched longitudinally, the result is a shape that can be more accurately described as an ellipsoid shape or a shape of a cylinder with convex outwardly curved base areas, depending on the degree of rounding/beveling of the proximal and distal domes and the stretching/compression of the basic body in the longitudinal direction. In this context, the base areas of the cylinder are considered to be the end faces. Depending on the intended application, the diameter or length of the basic body in the expanded state typically ranges between 4 and 25 mm. Such a diameter is sufficient to fill typical aneurysms, especially those located in the intracranial region. The diameter actually created within the aneurysm may vary, allowing the implant to be employed for the treatment of differently sized aneurysms and those having different sizes of the aneurysm neck.

Expediently, the implant is provided with one or several radiopaque markers allowing the attending physician to visualize the treatment. These, for example, may consist of a helix, spiral or rivet made of a radiopaque material, which are attached to the implant. Radiopaque markings on the microcatheter, as described above, are also useful. The radiopaque markers may, for example, consist of platinum, palladium, platinum-iridium, tantalum, gold, tungsten or other metals opaque to radiation. It is also possible to provide the implant, in particular the struts of the basic body with a coating consisting of a radiopaque material, for example with a gold coating. This coating can, for example, have a thickness of 1 to 6 μm. Coating with a radiopaque material need not be applied to the entire basic body. Nevertheless, even when applying a radiopaque coating it is considered useful to arrange one or several radiopaque markers on the implant, in particular at the distal end of the implant.

Another approach to render the implant radiopaque would be to embed radiopaque substances in the membrane, for example heavy metal salts such as barium sulfate. Such substances are known, for example, as contrast agents in X-ray technology applications.

An additional option includes using struts made of a metal having shape memory properties, especially an appropriate nickel-titanium alloy, which at least in part comprise a platinum core. Such struts are known as DFT (drawn filled tubing) wires. In this way, the advantageous properties of nickel-titanium on the one hand, namely imparting shape memory properties, are combined, on the other hand, with the beneficial properties offered by platinum, namely ensuring X-ray visibility.

The implant provided by the proposed invention is particularly suitable for the treatment of intracranial aneurysms, but its use for other types of aneurysms, for example aortic aneurysms or peripheral aneurysms, is also conceivable, in which case the dimensions of the implant are to be adapted as appropriate.

In addition to the implant itself, the invention also relates to the use of the implant for the treatment of arteriovenous deformities, in particular aneurysms. All statements made with reference to the implant itself also apply in an analogous manner to the use of the implant and to a method of using the implant.

Further elucidation of the invention is provided by way of examples through the enclosed figures. It is to be noted that the figures show preferred embodiment variants of the invention, with the invention itself not being limit thereto. To the extent it is technically expedient, the invention comprises, in particular, any optional combinations of the technical features that are stated in the claims or in the description as being relevant to the invention.

Clarification of the invention is provided by the following figures where

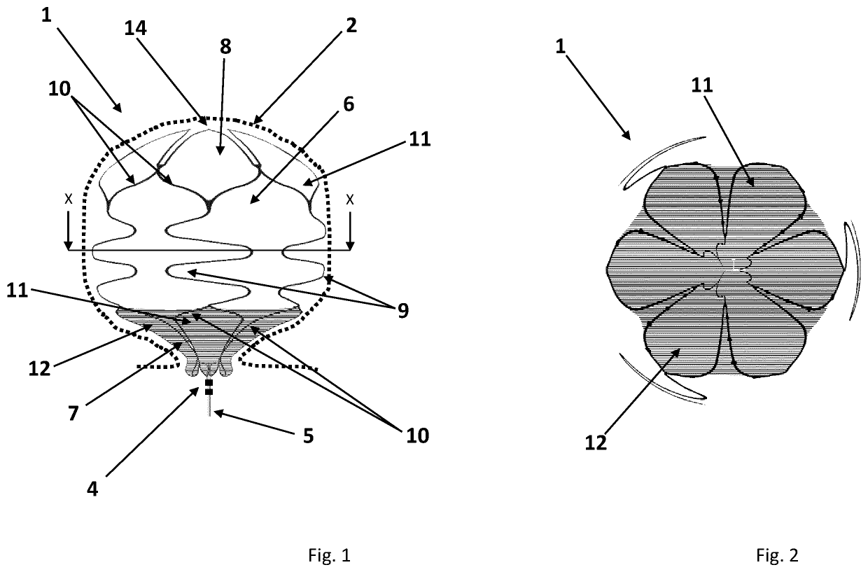

FIG. 1 shows a side view of an inventive implant in an aneurysm according to the first embodiment;

FIG. 2 shows the inventive implant from FIG. 1 in an X-X view from distal;

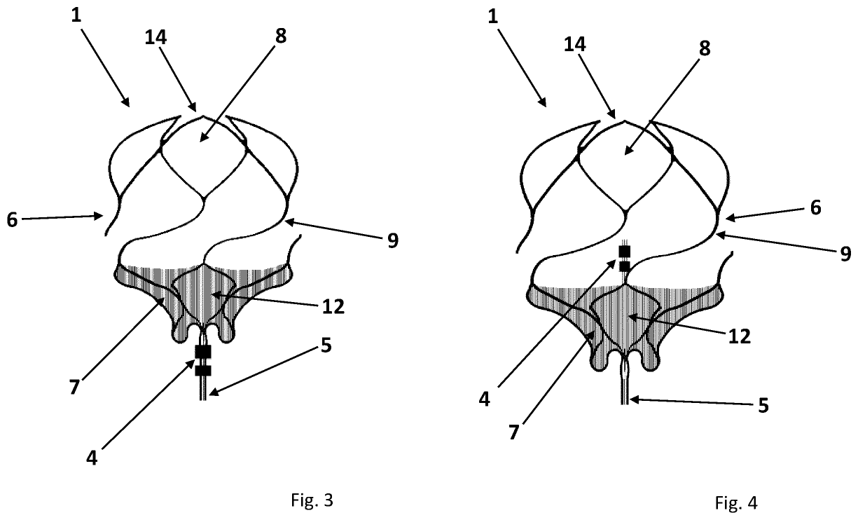

FIG. 3 is a side view of an inventive implant according to the first embodiment of the invention with a detachment point located proximal to the basic body;

FIG. 4 is a side view of an inventive implant according to the first embodiment of the invention with a detachment point arranged in the interior of the basic body;

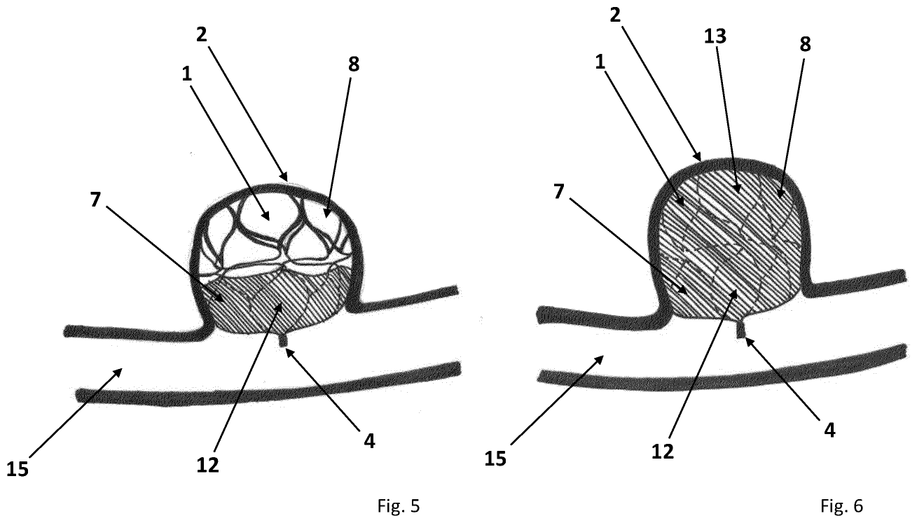

FIG. 5 shows an implant placed in an aneurysm according to the first embodiment with a membrane covering the proximal segment;

FIG. 6 shows an implant placed in an aneurysm according to the first embodiment with a membrane covering the proximal and the distal segment;

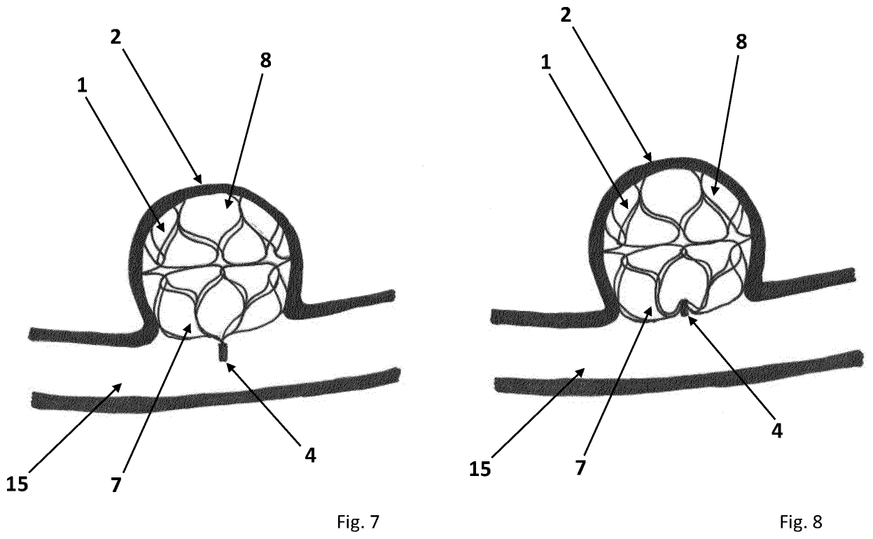

FIG. 7 shows an implant placed in an aneurysm according to the first embodiment without a membrane, with a detachment point being arranged proximal to the basic body;

FIG. 8 shows an implant placed in an aneurysm according to the first embodiment without a membrane, with a detachment point being arranged within the basic body;

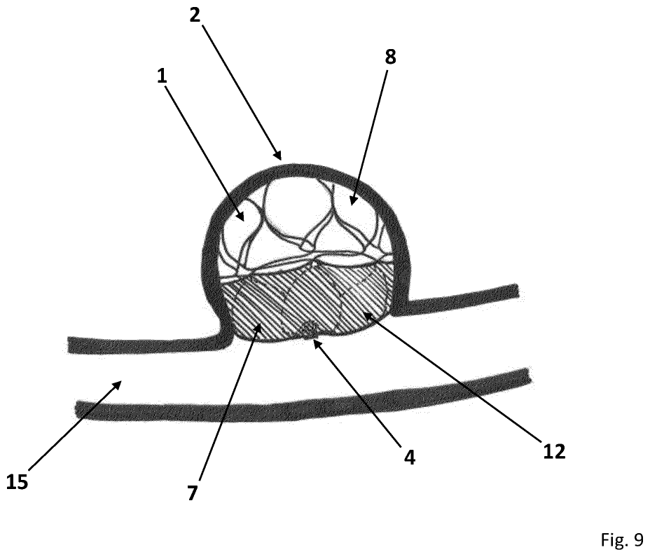

FIG. 9 shows an implant placed in an aneurysm according to the first embodiment with a membrane covering the proximal segment and with a detachment point being arranged in the interior of the basic body;

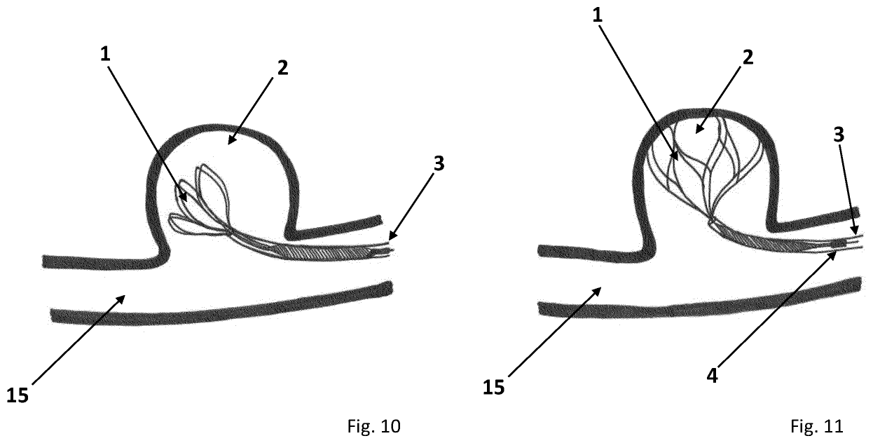

FIG. 10 shows an implant according to the second embodiment during its release inside the aneurysm;

FIG. 11 shows the release of the implant illustrated in FIG. 10 at a more advanced stage;

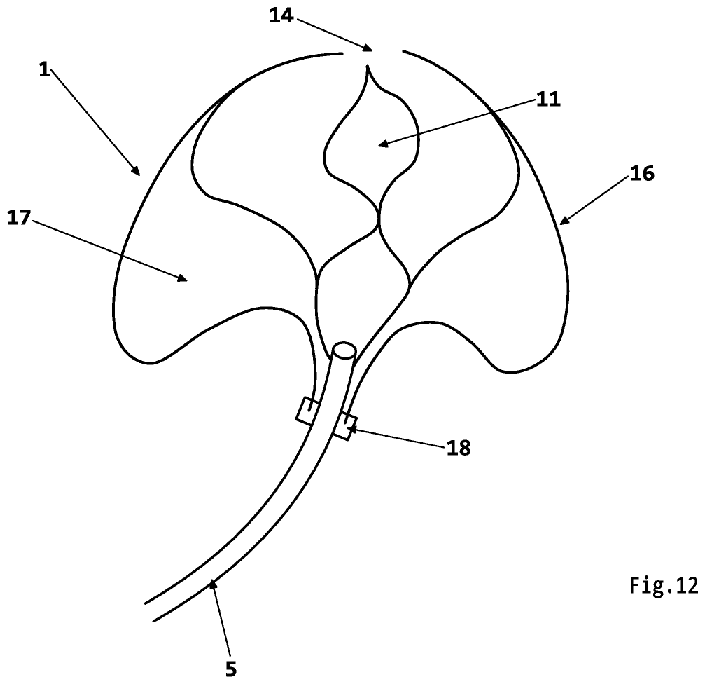

FIG. 12 depicts an implant according to the second embodiment including a tulip blossom-shaped basic body;

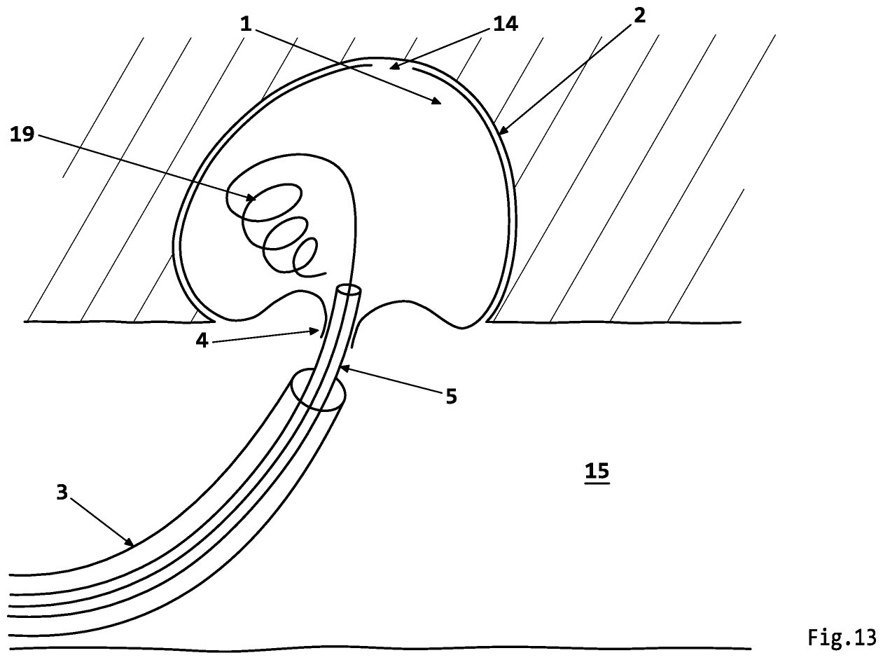

FIG. 13 shows the insertion of additional occlusion means into the implant illustrated in FIG. 12;

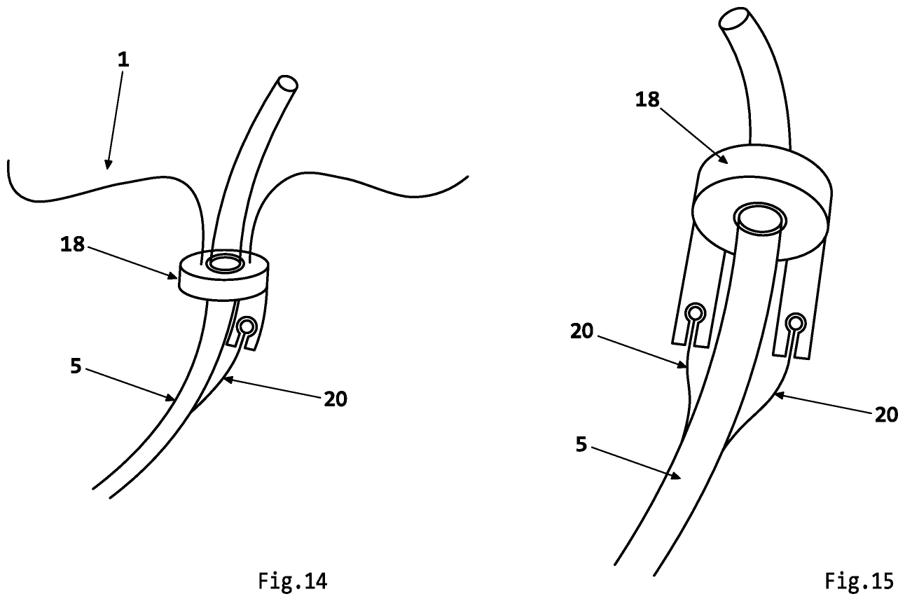

FIG. 14 shows an electrolytically corrodible detachment point for the severance of the implant;

FIG. 15 shows another electrolytically corrodible detachment point for the severance of the implant;

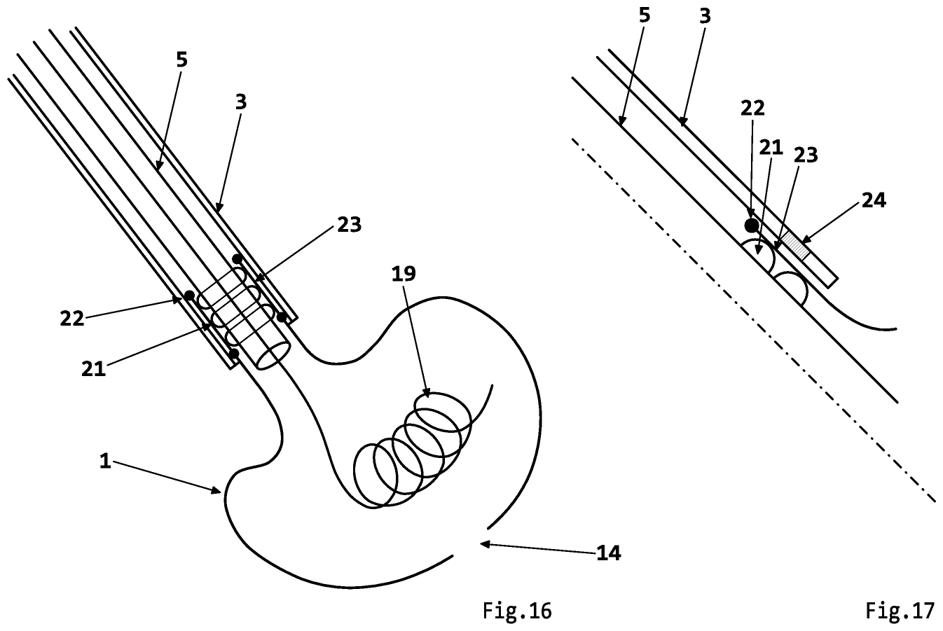

FIG. 16 shows a mechanically releasable, friction-fit based detachment point for release of the implant;

FIG. 17 is an enlarged view of the mechanically releasable detachment point for the release of the implant shown in FIG. 16;

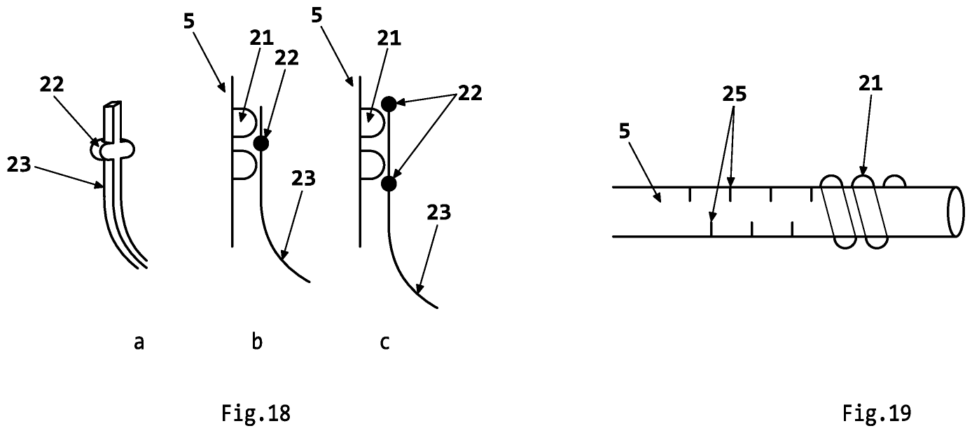

FIG. 18 illustrates further examples of mechanically releasable detachment points based on a friction-fit attachment;

FIG. 19 shows an insertion aid in the form of a flexible tube;

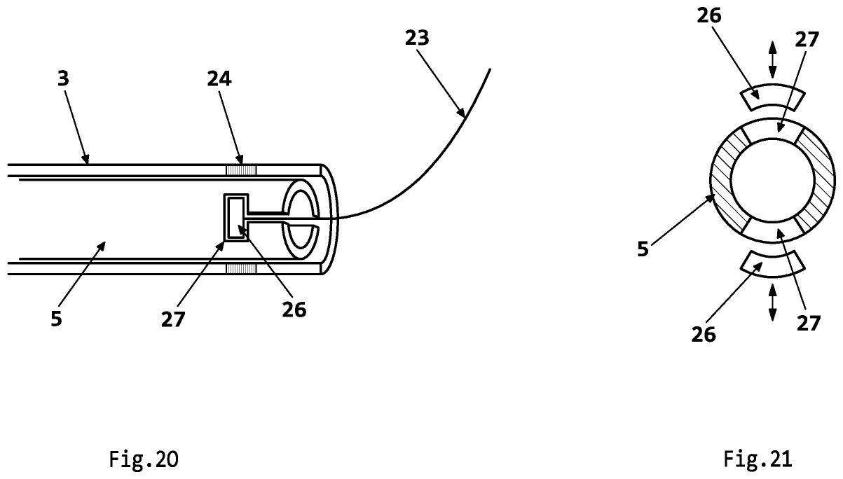

FIG. 20 shows a mechanically releasable, detachment point on form-closed basis for release of the implant;

FIG. 21 shows another mechanically releasable, detachment point on form-closed basis for release of the implant;

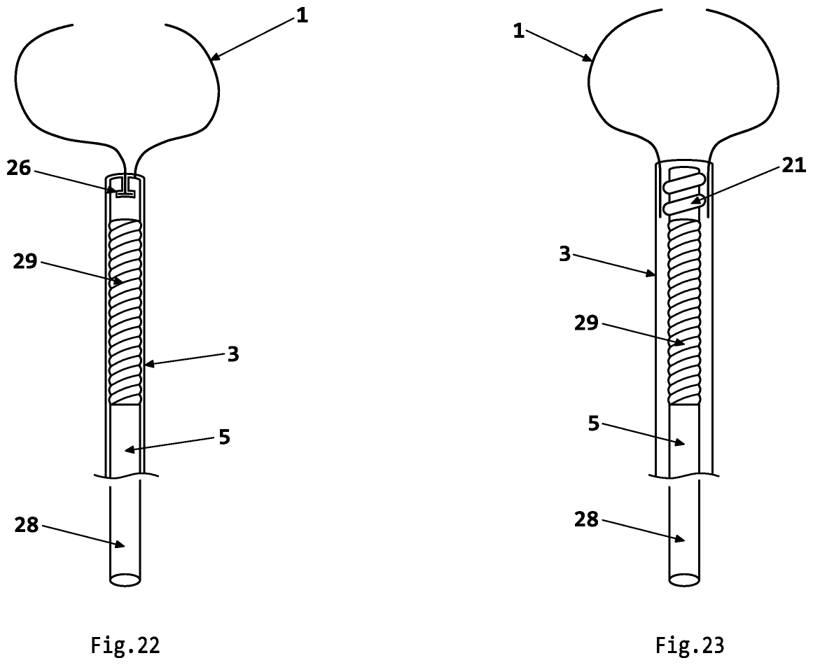

FIG. 22 shows an implant with a two-part insertion aid;

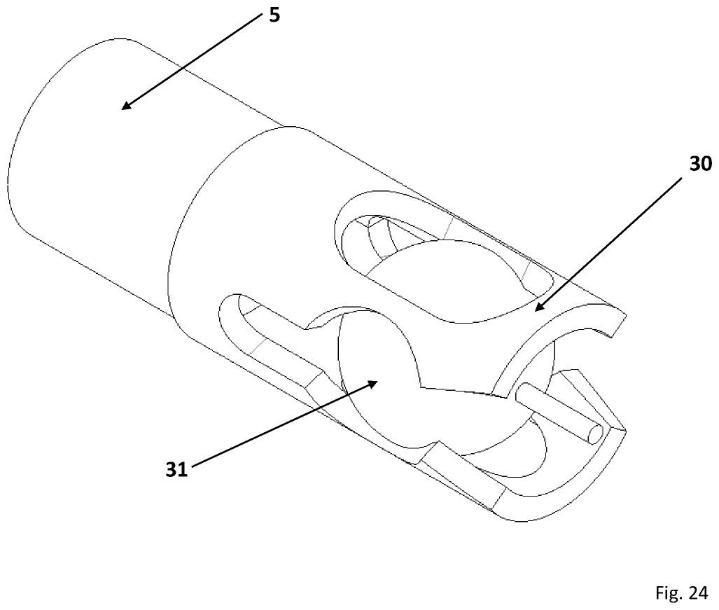

FIG. 23 shows another implant with a two-part insertion aid;

FIG. 24 is an oblique view of another variant of a detachment point on form-closed basis for detaching the implant in the closed state;

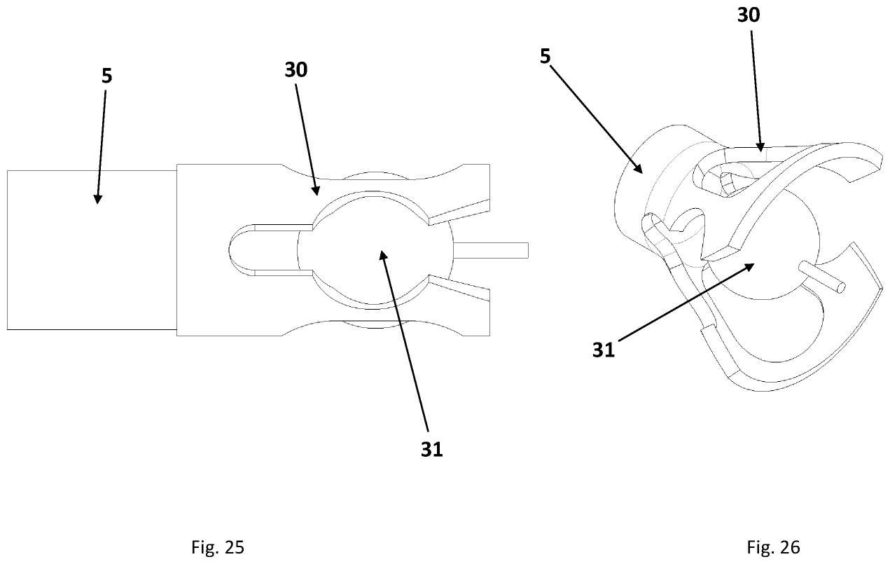

FIG. 25 is a side view of the detachment point from FIG. 24 in the closed state;

FIG. 26 is an oblique view of the detachment point from FIG. 24 in the open state;

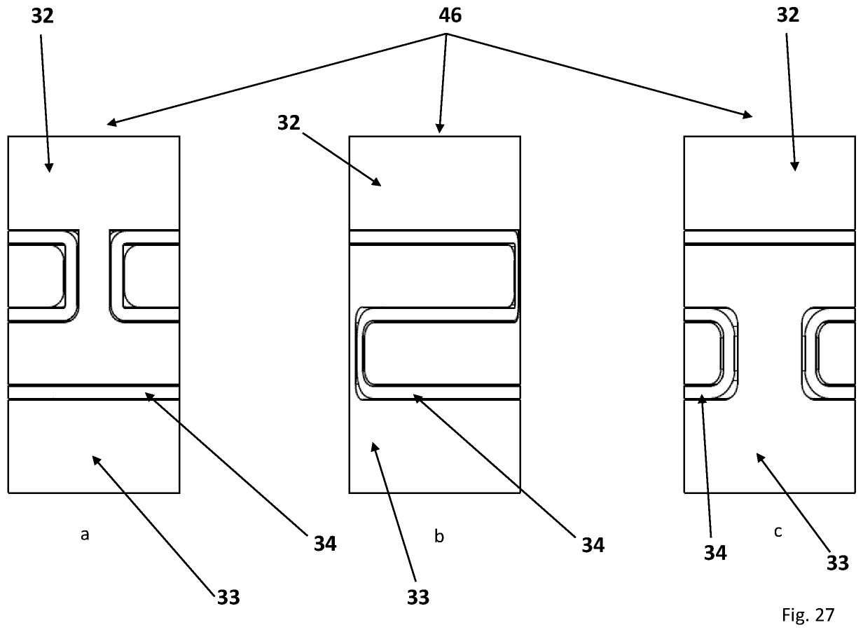

FIG. 27 shows a detachment element with a form-fit connection between the insertion aid and the implant, which allows electrolytic release;

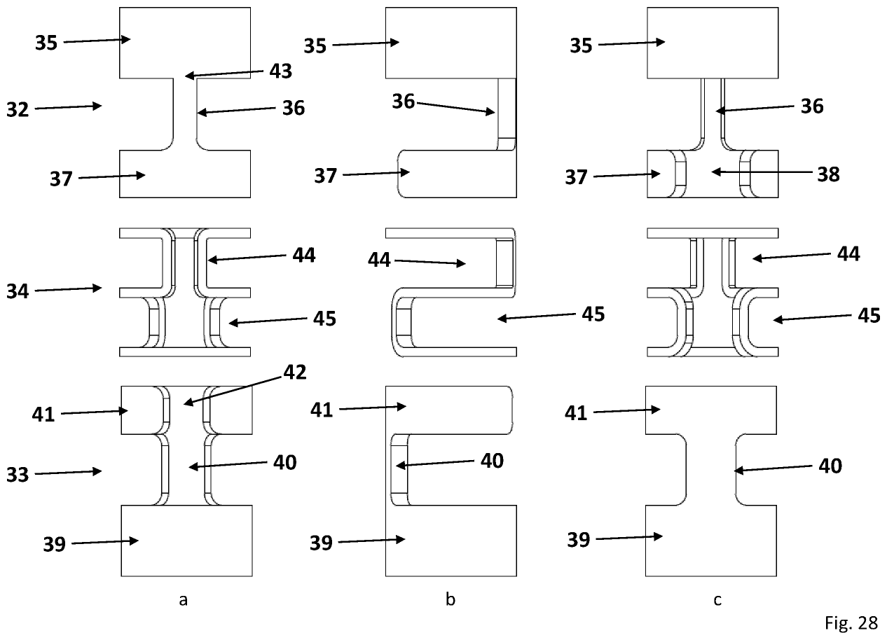

FIG. 28 shows the individual components of the detachment element from FIG. 27;

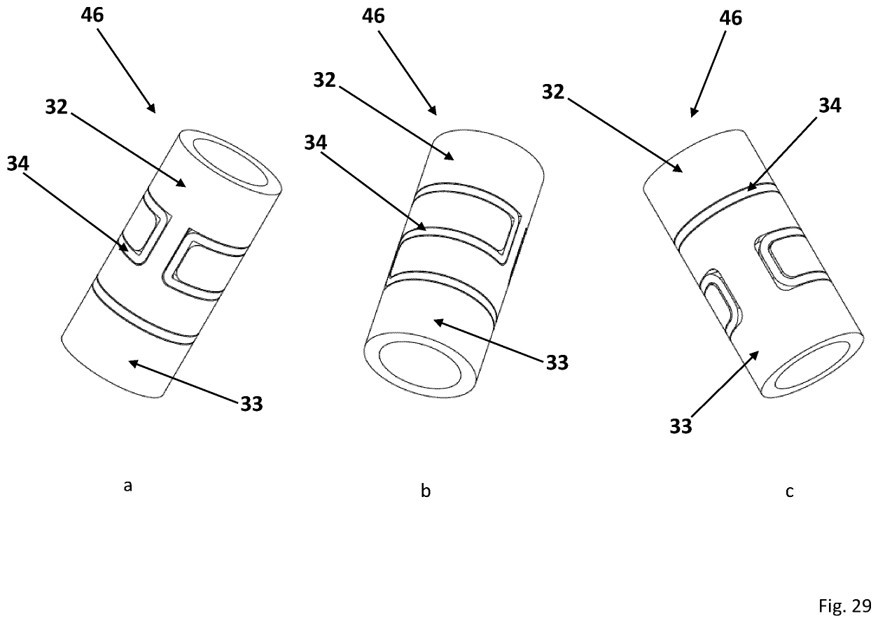

FIG. 29 shows different perspectives of the detachment element depicted in FIG. 27;

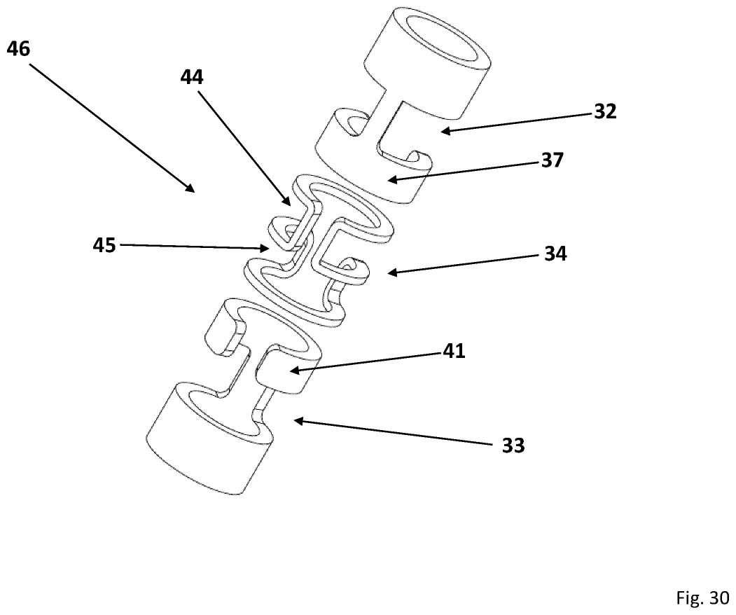

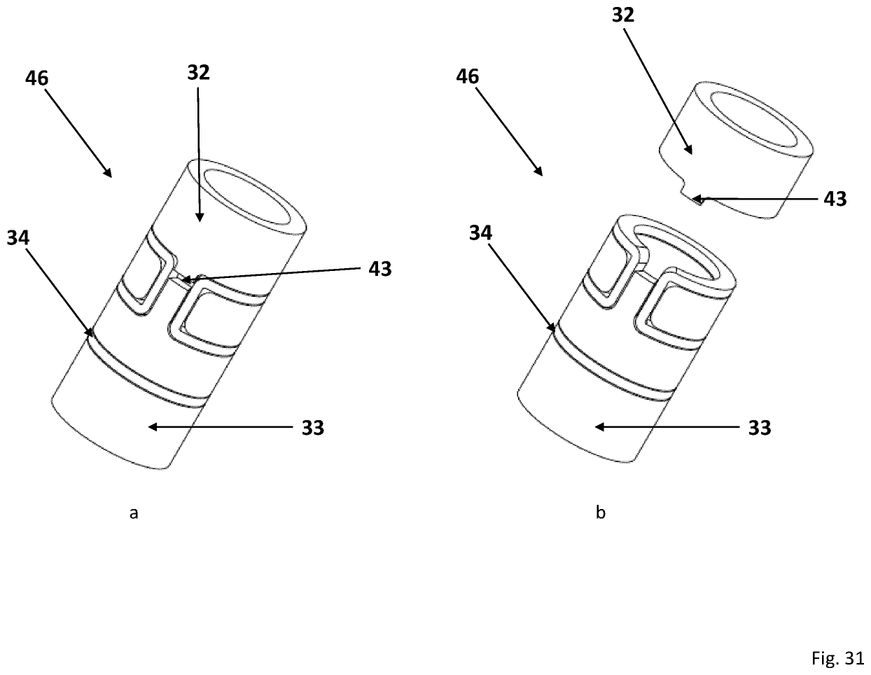

FIG. 30 is an exploded view of the interlocking components of the detachment element of FIG. 27 and

FIG. 31 shows the detachment of the implant using the detachment element illustrated in FIG. 27.

FIG. 1 is a representation of the invention according to the first embodiment shown as a side view. The Implant 1 has been placed in an aneurysm 2 signified by a dotted line. The implant 1 has a basic body 6, which is composed of a proximal segment 7, a distal segment 8 and connecting struts 9 by means of which the two segments are joined.

The proximal segment 7 is covered by a membrane 12. Both the proximal segment 7 and the distal segment 8 are made of frame struts 10 that form individual meshes or loops 11. In the proximal segment 7, the frame struts 10 are embedded in the membrane 12. The connecting struts 9 have a curvilinear shape, which ensures that the implant 1 can adapt well, both axially and radially, to the respective conditions of the aneurysm 2. This flexibility is further improved by an area 14 in the distal segment 8 which is free of frame struts 10.

In the proximal area, the implant 1 is connected to an insertion aid 5 via a detachment point 4, said insertion aid consisting of an insertion wire. The detachment point 4 may, for example, be designed to be detachable electrolytically, which enables the implant 1 to be detached after it has successfully been placed in the aneurysm 2.

In FIG. 2, implant 1 is shown as seen from the distal side according to the sectional view X-X. The individual loops 11 formed by the frame struts 10 can be seen, which are covered by a membrane 12. Membrane 12 has a blood flow modulating effect and causes the aneurysm 2 to be largely cut off from the flow of blood.

FIG. 3 is a side view of the first embodiment of the implant 1 proposed by the invention. The implant 1 again has a distal segment 8 and a proximal segment 7, which are connected to each other by connecting struts 9. The connecting struts 9 extend in a curved line. The proximal segment 7 is covered by a membrane 12. Further proximally, implant 1 is connected to the insertion aid 5 via the detachment point 4.

The illustration in FIG. 4 largely corresponds to that in FIG. 3, but in this case the detachment point 4 is provided inside the basic body 6. This ensures that after detachment of implant 1 no protruding tips of wire are left at the proximal end of implant 1.

In FIG. 5, implant 1 is shown in fully released state. In the present case, an implant 1 is shown in which the proximal segment 7 is covered by a membrane 12, whereas the distal segment 8 is not covered be a membrane. The detachment point 4 is arranged proximal to basic body 6.

FIG. 6 shows a corresponding implant 1, which deviates from the implant 1 of FIG. 5 in that the distal segment 8 in this representation also has been provided with a membrane 13. Further covering of the implant 1 with a membrane 13 offers the advantage that an inflow of blood into the aneurysm 2 is largely prevented even in the case of endoleaks.

FIG. 7 shows a corresponding implant 1 placed in the aneurysm 2 without membranes being provided here. Primarily, an implant 1 of this design serves as a supporting structure to retain occlusion means additionally introduced in the aneurysm 2.

FIG. 8 largely corresponds to FIG. 7, but unlike in FIG. 7, the detachment point 4 is arranged inside basic body 6. Accordingly, a central invagination/inward curvature of the basic body 6 results so that a protrusion of parts of the implant 1 into the main blood vessel 15 is ruled out.

In FIG. 9, another implant 1 is illustrated having a detachment point 4 arranged within basic body 6, in which the proximal segment 7 is covered by a membrane 12. The implant 1 thus largely corresponds to that in FIG. 5, but has a different arrangement of the detachment point 4.

In FIG. 10 a second embodiment of the inventive implant 1 is illustrated, which after expansion assumes the shape of a tulip blossom. The implant 1 is introduced into the aneurysm 2 through a microcatheter 3 via the blood vessel 15 and is liberated by advancing the implant 1 or retracting the microcatheter 3. In the state shown here, only the distal segment 8 of the implant 1 has unfolded, whereas areas of the implant 1 situated further proximal are still in the microcatheter 3.

In FIG. 11, the liberation of the implant 1 shown in FIG. 10 is in a more advanced stage. The detachment point 4 can be seen, but it is still located inside the microcatheter 3.

FIG. 12 shows a side view of the implant according to the second embodiment. The implant 1 is composed of partially interconnected struts 16, between which mesh-like interspaces 11 are formed. The implant 1 comprises a plurality of petal-shaped segments 17 that extend from the proximal end of implant 1, first radially outward and slightly in proximal direction, and then further distally and radially inward. In this way, the petal-shaped segments 17 adept closely to and against the inner wall of the aneurysm 2. A central area 14 remains without struts 16 at the distal end of the implant 1, resulting in an opening being created, the size of which may be greater or smaller as required by the size of the aneurysm 2.

At the proximal end, individual struts 16 of the implant 1 are connected to a detachment element 18 arranged in the form of a ring around the insertion aid 5. The insertion aid 5 is of tubular design and has an inner lumen through which additional occlusion means can be introduced into the implant 1. A release of the implant 1 can be brought about by subjecting the annular detachment element 18 to electrolytic corrosion. In particular, implant 1 can initially be inserted into the aneurysm 2 where it is caused to expand without any release having already occurred at the detachment element 18. Further occlusion means can then be introduced through the insertion aid 5 to fill the interior space of the implant 1. Subsequently, when this process is complete, an electrical current is applied to the ring-shaped detachment element 18 to cause the implant 1 to be liberated. Finally, insertion aid 5 and the microcatheter 3 which is not shown here, are retracted proximally and removed from the blood vessel system.

FIG. 13 illustrates how through insertion aid 5 additional occlusion means 19 are introduced into the interior space of the implant 1. Implant 1 corresponds to the implant shown in FIG. 12, but is displayed here in a simplified form. By retracting the microcatheter 3, the implant 1 has already been expanded in the aneurysm 2, causing the struts 16 of the implant 1 to be placed in position against the inner wall of aneurysm 2. However, the detachment of implant 1 has not yet occurred.

The FIGS. 14 and 15 show different ways of detaching implant 1 electrolytically. In both cases, a ring-shaped detachment element 18 is arranged around the tubular insertion aid 5, said element being in connection with the proximal end of the implant 1, which is only hinted at here. An electrical current can be applied to the detachment element 18 via one (FIG. 14) or two (FIG. 15) electrically conductive detachment wires 20, which enables electrolytic corrosion to be initiated, causing detachment and final liberation of the implant 1. Detachment wires 20 are appropriately insulated so that the electric current is applied in a precisely targeted way to the detachment point. Alternatively, the detachment wire 20 may extend through the lumen of the insertion aid 5, or the metal mesh of a catheter-like insertion aid may serve as a suitable conductor.

In FIGS. 16 and 17 a detachable connection of the implant 1 to the insertion aid 5 is illustrated, which is based on frictional engagement. Radial projections 21 in the form of elastic pads are arranged around the internally hollow insertion aid 5. The proximal end 23 of the implant 1, which in this case comprises individual struts of the implant 1 projecting in proximal direction, has been provided with spherical thickenings 22 and is clamped between the microcatheter 3 and the insertion aid 5. Interaction of the microcatheter 3, the insertion aid 5, the proximal end 23 of the implant 1 and, in particular, the radial protrusions 21 and the thickenings 22 results in a frictional connection being established and rules out premature detachment of implant 1.

FIG. 17 shows the principle in detail. As soon as microcatheter 3 is retracted in proximal direction (top left in this representation), the proximal end 23 of the implant 1 can expand radially causing the implant 1 to be detached. To enable the treating physician to check the detachment process, radiopaque markers 24 are additionally provided on microcatheter 3.

FIG. 18a, b, c shows further mechanically separable detachment points based on frictional locking, with a combination of frictional locking and form-closed locking being brought about by appropriate thickenings 22 arranged at the proximal end 23 of implant 1. The thickenings 22 of the proximal end 23 of the implant 1 are arranged either between the radial projections 21 of the insertion aid 5 or vice versa. In each case a microcatheter 3, not shown here, surrounds the arrangement and keeps the proximal end 23 from expanding in radial direction. Accordingly, detachment of implant 1 cannot occur until microcatheter 3 has been retracted in proximal direction.

In FIG. 19 a tubular insertion aid 5 is shown on which radial protrusions 21 are arranged to bring about the frictional connection. Additionally, various slits 25 are provided in the insertion aid 5 that increase flexibility and facilitate the advancement of the implant 1 through the microcatheter 3, particularly in narrow blood vessels.

FIG. 20 depicts a form-closed fastening of the proximal end 23 of the implant 1. Form-closed detachment elements 26 are arranged at the proximal end 23, which engage in corresponding recesses 27 in the insertion aid 5. When the microcatheter 3 is retracted in proximal direction, the form-closed detachment elements 26 can expand radially and exit the recesses 27, resulting in the release of the implant 1. To allow the retraction process of the microcatheter 3 to be visualized, the catheter also is provided with radiopaque markers 24.

Another example of form-closed detachment elements 26 is shown in FIG. 21, with said detachment elements 26 being shaped like segments of a circle and designed to fit into corresponding recesses 27 arranged in the insertion aid 5. When the microcatheter 3 is retracted, the detachment elements 26 can exit the recesses 27, as indicated by the arrows.

In FIGS. 22 and 23, it is shown that the insertion aid 5 can be composed of two parts. FIGS. 22 and 23 are basically identical, but FIG. 22 shows a form-closed detachment and FIG. 23 shows a frictional detachment as described hereinbefore. The insertion aid 5, which has an inner lumen, is more rigidly formed in the proximal part 28 than in the more flexible distal part 29. For this purpose, the distal part 29 has a helical structure in the form of a two-layer hollow strand that has a high degree of flexibility. In order to be able to transmit torsional forces as well, the turns of the helical structure are opposed to each other.

FIG. 24 depicts a further variant for fastening the implant 1 to the insertion aid 5 via a form-closed connection, in which at least one detachment element 31 extends in the proximal direction from the proximal end of the implant 1 and is held in a form-closed manner by a retaining element 30 arranged on the insertion aid 5, said retaining element thus engaging around the detachment element 31. The detachment element 31 has a spherical shape. The retaining element 30 is made of a material having shape memory properties. Microcatheter 3, which is not illustrated here, is arranged around the retaining element 30 in the closed state and prevents the latter from assuming an expanded secondary structure and releasing the detachment element 31. FIG. 25 is a side view of the same situation.

FIG. 26 shows the situation after the microcatheter 3 has been retracted in the proximal direction. The retaining element 30 takes on the preset open secondary structure so that the detaching element 31 can exit which thus causes in the implant 1 to be detached.

FIG. 27 represents a detachment element 46 based on a combination of mechanical form-closed locking and electrolytic detachability. The detachment element 46 is shown from the front (a), side (b) and rear (c). The detachment element 46 is composed of 3 components, the insertion aid end piece 32, which is located at the distal end of the insertion aid not shown here, the implant end piece 33, which is located at the proximal end of the implant not shown here, and the insulating element 34, which electrically separates the insertion aid end piece 32 and the implant end piece 33 from each other, even when they are in engagement with each other. The insertion aid end piece 32 and the implant end piece 33 overlap, i.e. the distal area of the insertion aid end piece 32 is located distal to the proximal area of the implant end piece 33.

In FIG. 28, the individual components of the detachment element 46 are shown from the front (a), from the side (b) and from the rear (c). The insertion aid end piece 32 consists of a first short tubular member 35 that is closed radially. This is joined via a connecting web 36 to a second short tubular element 37, which has a radial interruption 38.

The implant end piece 33 has a virtually identical structure, i.e., it has a radially closed first short tubular element 39, a second short tubular element 41 radially provided with an interruption 42, and a connecting web 40 arranged between the two. However, the position of the insertion aid end piece 32 and the implant end piece 33 is inverted, i.e. the second tubular elements 37 and 41 provided with interruptions 38, 42 face each other, with the interruptions 38, 42 having an offset of 180°.

The insulating element 34 has a proximal portion 44 and a distal portion 45, and the second tubular element 37 of the insertion aid end piece 32 engages with and surrounds the distal portion 45, whereas the second tubular element 41 of the implant end piece 33 engages with and surrounds the proximal portion 44. In this context, the recesses existing in the proximal and distal sections 44, 45 are in alignment with the second tubular elements 37, 41, i.e., the recesses are completely or largely filled by them. It is achieved in this way that the insertion aid end piece 32 and the implant end piece 33 interlock with each other, but an area of the insulating element 34 is always arranged between the two, so that a voltage applied to the insertion aid end piece 32 is not transmitted to the implant end piece 33, resulting in the two end pieces 32, 33 being insulated from each other.

Also of importance is the electrolytically corrodible detachment point 43, which dissolves when a current is applied so that separation occurs at this point. The implant is thus released together with the parts of the detachment element 46 that still remain attached, while the proximally located first tubular element 35 of the insertion aid end piece 32 remains on the insertion aid and is retracted in the proximal direction with it. The detachment point 43 is the narrow connection point between the first tubular element 35 and the connecting web 36.

FIG. 29 shows the interconnected detachment element 46 with insertion aid end piece 32, implant end piece 33 and insulating element 34 from different perspectives. It can be seen how insertion aid end piece 32 and implant end piece 33 are engaged with each other in a form-closed manner, but areas of insulating element 34 always prevent direct contact between insertion aid end piece 32 and implant end piece 33. Furthermore, it can also be seen that a lumen of the detachment element 46 is provided for connection to the lumen of the tubular or hose-shaped insertion aid. Accordingly, this enables additional occlusion means such as coils or embolization agents to be introduced into the aneurysm.

FIG. 30 is an exploded view of the detachment element 46, from which it can be seen that the second tubular elements 37 and 41 of the insertion end piece 32 and implant end piece 33, which are provided with an interruption, overlap one another, with insulating element 34 being located in between. Both the interruptions 38, 42 in the end pieces 32, 33 and the recesses in the insulating element 34, which are designed to fit to the second tubular elements 37, 41, are radially offset by 180°.

Finally, FIG. 31 shows the detachment of the implant at the detachment element 46. A current is applied to the detachment point 43 via the insertion aid and the metallic insertion aid end piece 32, with said detachment point 43 being connected as an anode. This arrangement causes the corrodibly designed detachment point 43 to dissolve and the proximal area of the insertion aid end piece 32 disconnects from the other areas of the detachment element 46. The latter are released together with the implant, while the proximal portion of the insertion aid end piece can be retracted in proximal direction and removed from the blood vessel system. The metallic portions of the detachment element 46 remaining on the implant may be made of magnesium, for example, which dissolves over time, causing foreign objects that are no longer needed to disappear. The detachment element 46 ensures a secure form-closed connection between the insertion aid and the implant, which remains in place even if a surrounding microcatheter is retracted proximally resulting in the detachment element 46 to be liberated. Release of the implant does not occur until a voltage is applied to the detachment point 43. Moreover, the form-closed connection between the insertion aid and the implant also allows the transmission of torsional movements.

In the claims:

1. An implant for the treatment aneurysms (2), wherein the implant (1) can be brought in a compressed state through a microcatheter (3) to a target site in the blood vessel system of a patient, with the implant (1) being preset to a secondary structure causing it to assume an expanded state when released from the microcatheter (3), wherein the implant (1) is separably connected to an insertion aid (5) at a detachment point (4), wherein:
the implant (1) in the expanded state has a basic body (6) comprising of a proximal and a distal segment (7, 8), with the proximal and the distal segment (7, 8) being of dome-shaped configuration, with the convex side of the dome of the proximal segment (7) facing in the proximal direction and the convex side of the dome of the distal segment (8) facing in the distal direction, and wherein the proximal and the distal segment (7, 8) are connected to each other via a plurality of connecting struts (9).

2. An implant according to claim 1, wherein the configuration of the connecting struts (9) arranged between proximal and distal segments (7, 8) is curvilinear.

3. An implant according to claim 1, wherein the proximal and/or the distal segment (7, 8) are constructed from frame struts (10) which are at least partially connected to each other.

4. An implant according to claim 3, wherein in the expanded state the frame struts (10) form a mesh or loop structure (11) in the proximal and/or distal segment (7, 8).

5. An implant according to claim 3, wherein the distal segment (8) preferably comprises centrally an area (14) free of frame struts (10), said area (14) being expandable and compressible.

6. An implant according to claim 1, wherein the proximal segment (7) is provided with a membrane (12) which covers the proximal segment (7) at least partially.

7. An implant according to claim 6, wherein the distal segment (8) is provided with a membrane (13) which covers the distal segment (8) at least partially.

8. An implant according to claim 6, wherein the connecting struts (9) are at least partially covered with a membrane.

9. An implant for the treatment of aneurysms (2), with the implant (1) being navigable in a compressed state via a microcatheter (3) to a destination in the blood vessel system of a patient, and with the implant (1) being preset to a secondary structure causing it to assume an expanded state when released from the microcatheter (3), with the implant (1) being detachably connected to an insertion aid (5) via a detachment point (4) and, in the expanded state, having a basic body (6) which is composed of struts (16), wherein
said struts (16) being at least partially connected to one another at points of intersection, so that interspaces (11) are created between the struts (16), with the struts (16), in the expanded state, extending radially outwards at the proximal end of the basic body (6) and, in the further course, extending axially in the distal direction and radially inwards, so that bulging of the basic body (6) is produced, with the basic body (6) having a zone, at the distal end, where the struts (16) do not have any connection to one another.

10. An implant according to claim 9, wherein the basic body (6) in the expanded state has an opening (14) at the distal end.

11. An implant according to claim 1, wherein the basic body (6) in the expanded state has, by approximation, a spherical shape, an ellipsoidal shape, an ovoid shape, a tulip- blossom shape or the shape of a cylinder with convex outwardly curved base surfaces.

12. An implant according to claim 1, wherein the distal end of the insertion aid (5) is located in the interior of the basic body (6) when the latter is in the expanded state.

13. An implant according to claim 1, wherein the insertion aid (5) is of tubular or hose-shaped design and has an inner lumen.

14. An implant according to claim 13, wherein the tubular or hose-shaped insertion aid (5) projects in distal direction beyond the detachment point (4).

15. An implant according to claim 1, wherein a detachment element (18) is arranged on the outside of the insertion aid (5), said element being connected to the proximal end (23) of the implant (1), wherein by applying an electrical voltage to the detachment element (18) a liberation of the implant (1) can be brought about.

16. An implant according to claim 15, wherein the detachment element (18) is arranged annularly around the insertion aid (5).

17. An implant according to claim 1, wherein the insertion aid (5) has radial protrusions (21) at its distal end on the outside and projects with the distal end into the implant (1) so that a frictional connection between the insertion aid (5), the implant (1) and the microcatheter (3) or a sheath surrounding the distal end of the insertion aid (5) is provided, with the implant (1) being liberated by a movement of the microcatheter (3) or the sheath in the proximal direction relative to the implant (1) and the insertion aid.

18. An implant according to claim 17, wherein the radial protrusions (21) are made of an elastic material.

19. An implant according to claim 13, wherein detachment elements (26) are arranged at the proximal end of the implant (1), said detachment elements engage in a form-closed manner in recesses (27) provided for this purpose in the tubular or hose-shaped insertion aid (5), so that detachment of the implant (1) takes place by movement of the microcatheter (3) or of a sheath surrounding the distal end of the insertion aid (5) in the proximal direction relative to the implant (1) and to the insertion aid (5).

20. An implant according to claim 1, wherein at least one detachment element (31) extends in the proximal direction at the proximal end of the implant (1), said element being secured in a form-closed manner by a retaining element (30) arranged on the insertion aid (5), wherein the retaining element (30) being made of a material having shape memory properties and with the retaining element (30) being preset to a secondary structure, the detachment element (31) being released and detachment of the implant (1) taking place when the secondary structure is assumed, wherein the retaining element (30) is prevented from assuming the secondary structure by the surrounding microcatheter (3) or another sheath surrounding the retaining element (30).

21. An implant according to claim 1, wherein an insertion aid end piece (32) is arranged at the distal end of the insertion aid (5), said end piece having an electrolytically corrodible detachment point (43), wherein the insertion aid end piece (32) is connected in a form-closed manner to an implant end piece (33) arranged at the proximal end of the implant (1), and wherein an insulating element (34) is arranged between the insertion aid end piece (32) and the implant end piece (33) in such a way that direct contact between the insertion aid end piece (32) and the implant end piece (33) is avoided.

22. An implant according to claim 21, wherein the insertion aid end piece (32) and the implant end piece (33) each have a first tubular element (35, 39) and a second tubular element (37, 41), a connecting web (36, 40) is arranged between each the first tubular element (35, 39) and the second tubular element (37, 41), and the second tubular elements (37, 41) each have a radial interruption (38, 42), wherein the insertion aid end piece (32) and the implant end piece (33) are joined together in such a way that the second tubular element (41) of the implant end piece (33) is arranged proximally to the second tubular element (37) of the insertion aid end piece (33).
